(19) United States
(12) Patent Application Publication
(10) Pub. No.: US 2026/0000138 A1
(43) Pub. Date: Jan. 1, 2026

(54) Elastomeric Article with Imprinted Pattern on One Side

(71) Applicant: O&M Halyard, Inc., Glen Allen, VA (US)

(72) Inventors: Natalie Reeves, Mableton, GA (US); Marshall R. Dean, Cumming, GA (US); Tantima Saelim, Songkhla (TH); Shantilal H. Modha, Milton, GA (US); Siew Hoe Tan, Penang (MY); Rossarin Jivanit, Songkhla (TH)

(21) Appl. No.: 19/250,281

(22) Filed: Jun. 26, 2025

Related U.S. Application Data
(60) Provisional application No. 63/664,450, filed on Jun. 26, 2024.

(51) Int. Cl. A41D 19/015; B29C 41/14; B29C 41/38; B29K 105/00; B29L 31/48

(52) U.S. Cl. CPC A41D 19/01547; B29C 41/14; B29C 41/38; B29K 2105/0058; B29L 2031/4864

(57) ABSTRACT

An elastomeric article and a process for forming an elastomeric article are provided. The elastomeric article includes a body having a first side and a second side opposite the first side. The body is formed from an elastomeric material including polyurethane, nitrile rubber, styrene-butadiene rubber, isobutylene-isoprene rubber, polychloroprene, polyisoprene, natural rubber, or a combination thereof. The first side includes a uniform surface. The second side includes a surface having an embossed shape extending therefrom in a direction opposite the first side of the body.

### RELATED APPLICATIONS

The present application claims priority to U.S. Provisional Application Ser. No. 63/664,450, filed on Jun. 26, 2024, which is incorporated herein in its entirety by reference thereto.

### FIELD OF THE INVENTION

The subject matter of the present disclosure relates generally to elastomeric articles that have an imprinted or textured pattern on one side of the elastomeric article. In particular, the invention pertains to any elastomeric glove (e.g., surgical, exam, industrial, emergency responder, etc.) containing an imprinted or textured pattern on one side thereof.

### BACKGROUND

The development of modern rubber materials has made possible the manufacture of a wide range of elastomeric articles, such as gloves, having varying properties of strength and chemical resistance. Gloves are used as an infection protection device to protect the wearer from exposure to bacteria, viruses, pathogens, infections, diseases, etc. that could transfer from a surface or bodily fluid (e.g., blood) to the wearer's skin. Gloves are also used in manufacturing environments to prevent the wearer from coming into contact with various chemicals, and in some medical settings, gloves can be used to protect the wearer from certain pharmaceuticals that may be toxic, such as chemotherapy drugs.

While the elastomeric material provides excellent protection to the wearer against exposure to undesired substances, existing elastomeric gloves have a generally smooth surface on both the inner (donning) side of the glove and the outer (grip) side of the glove. In some settings, such as when a user is holding certain instruments or tools during a manufacturing setting, the smooth surface may not provide sufficient friction for gripping the tool or instrument.

Accordingly, improved elastomeric articles having enhanced gripping features are desired in the art. In particular, elastomeric articles such as gloves which provide imprinted pattern or texture on one side, such as an outer side of a glove, would be advantageous.

### SUMMARY

Objects and advantages of the invention will be set forth in part in the following description, or may be obvious from the description, or may be learned through practice of the invention.

An elastomeric article includes a body having a first side and a second side opposite the first side, the body being formed from an elastomeric material. The elastomeric material includes polyurethane, nitrile rubber, styrene-butadiene rubber, isobutylene-isoprene rubber, polychloroprene, polyisoprene, natural rubber, or a combination thereof. The first side comprises a uniform surface, and the second side comprises a surface having an embossed shape extending therefrom in a direction opposite the first side of the body.

The elastomeric article of any one or more of the embodiments, wherein the surface of the second side includes a repeating pattern of the embossed shape.

The elastomeric article of any one or more of the embodiments, wherein the body forms a glove.

The elastomeric article of any one or more of the embodiments, wherein the first side forms an inside surface of the glove and the second side forms an outside surface of the glove.

The elastomeric article of any one or more of the embodiments, wherein the surface of the second side comprises a repeating pattern of the embossed shape.

The elastomeric article of any one or more of the embodiments, wherein the repeating pattern of the embossed shape extends on both a palm side of the outside surface of the glove and a dorsal side of the outside surface of the glove.

The elastomeric article of any one or more of the embodiments, wherein the glove has a palm region thickness ranging from about 0.01 millimeters to about 6 millimeters.

The elastomeric article of any one or more of the embodiments, wherein the embossed shape comprises at least one alphabetical letter.

The elastomeric article of any one or more of the embodiments, wherein the at least one alphabetical letter comprises a plurality of letters forming a word or an acronym.

The elastomeric article of any one or more of the embodiments, wherein the uniform surface of the first side is smooth.

The elastomeric article of any one or more of the embodiments, wherein the uniform surface is free from any embossed or debossed shapes.

The elastomeric article of any one or more of the embodiments, wherein the body comprises a first layer and a second layer, wherein the first layer forms the first side of the body and the second layer forms the second side of the body.

The elastomeric article of any one or more of the embodiments, wherein the body has a first thickness extending from the first side to the second side, wherein the body has a second thickness extending from the first side to the embossed shape of the second side, the second thickness being greater than the first thickness.

A process for making an elastomeric article, the process comprising steps of: providing a mold having a former surface, the mold having a shape debossed in the former surface; dipping the mold into a first solution comprising a first powder free coagulant; dipping the mold into a first elastomeric formulation comprising a first elastomeric material to form a first layer; and curing the first elastomeric formulation to form the elastomeric article, the elastomeric article having a mold side in contact with the mold and a free side opposite the mold side; and removing the elastomeric article from the mold by inverting the elastomeric article such that the free side of the elastomeric article forms an inside surface of the elastomeric article and the mold side of the elastomeric article forms an outside surface of the elastomeric article, the mold side of the elastomeric article comprising a surface and the shape embossed on the surface.

The process of any one or more of the embodiments, wherein after the step of dipping the mold into the first elastomeric formulation, the process includes steps of dipping the mold into a second solution comprising a second powder free coagulant; and dipping the mold into a second elastomeric formulation comprising a second elastomeric material to form a second layer.

The process of any one or more of the embodiments, wherein the shape debossed in the former surface is a mirror image of the shape embossed on the surface of the elastomeric article.

The process of any one or more of the embodiments, wherein the inside surface of the elastomeric article comprises a uniform surface.

The process of any one or more of the embodiments, wherein the uniform surface is free from any embossed or debossed shapes.

The process of any one or more of the embodiments, further comprising a step of printing a visual marking on the shape embossed on the surface.

The process of any one or more of the embodiments, further comprising a step of printing a visual marking on the free side of the elastomeric article.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of an elastomeric article according to one particular embodiment of the present invention;

FIG. 2 illustrates a cross-sectional view of the elastomeric article of FIG. 1 taken along the line 1-1;

FIG. 3 illustrates a mold for forming the elastomeric article of FIG. 1 according to one particular embodiment of the present invention; and

FIG. 4 illustrates a mold for forming an elastomeric article according to one particular embodiment of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents. The detailed description uses numerical and letter designations to refer to features in the drawings. Like or similar designations in the drawings and description have been used to refer to like or similar parts of the invention.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Terms of approximation, such as “about,” “generally,” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

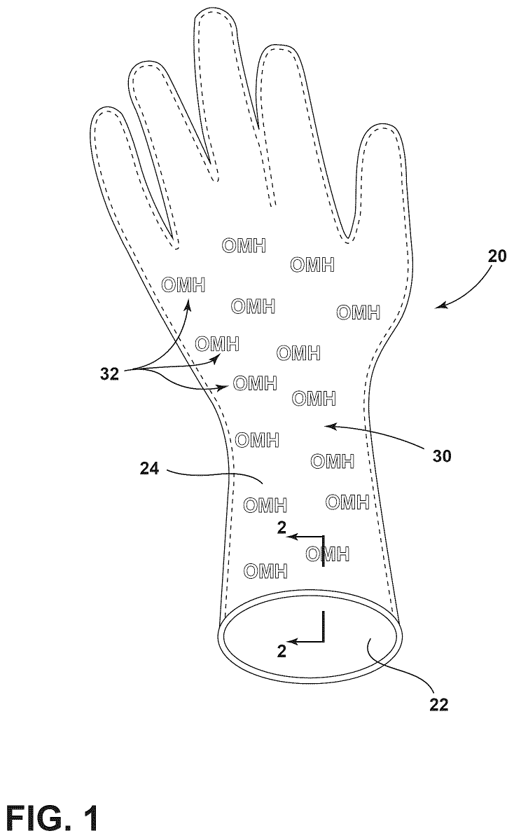

Generally speaking, the present invention is directed to an elastomeric article and a process for forming an elastomeric article are provided. The elastomeric article includes a body having a first side and a second side opposite the first side. The body is formed from an elastomeric material including polyurethane, nitrile rubber, styrene-butadiene rubber, isobutylene-isoprene rubber, polychloroprene, polyisoprene, natural rubber, or a combination thereof. The first side includes a uniform surface. The second side includes a surface having an embossed shape extending therefrom in a direction opposite the first side of the body. The embossed shape on the elastomeric article is formed by debossing a mirror of the embossed shape into a mold or former on which the article is formed. The elastomeric article may be an elastomeric glove. The present inventors have found that providing embossed shapes on the outside surface of the elastomeric article, e.g., glove, may augment the gripping surface of the glove by increasing friction during gripping. The specific features of the elastomeric article of the present invention may be better understood with reference to FIGS. 1-3.

Referring now to FIG. 1, one embodiment of an elastomeric article 20 made according to the present disclosure is illustrated. The elastomeric article 20 generally includes a first surface 22 and a second surface 24. As shown in FIG. 1, the elastomeric article 20 may be, for example, a glove. The first surface 22 may generally include an inside surface of the glove, and the second surface 24 may generally be an outside surface of the glove. As used herein, the “inside surface” refers to the surface of the article that contacts the body of the wearer. As used herein, the “outside surface” refers to the surface of the article that is distal from the body of the wearer. The article 20 includes a substrate body 26. As used herein, “first surface” refers to the surface of the substrate body proximal to the body of the wearer. As used herein, “second surface” refers to the surface of the substrate body distal to the body of the wearer.

The article of the present disclosure may include a single layer or multiple layers as desired. In a single layer glove including only the substrate body, the first surface may form the inside surface of the glove. However, in a multi-layer glove having additional layers proximal the body of the wearer, the additional layer or layers may each form a portion of the inside surface, or the entire inside surface, as desired. Likewise, in a single layer glove including only the substrate body, the second surface may form the outside surface of the glove. However, in a multi-layer glove having additional layers distal from the body of the wearer, the additional layer or layers may each form a portion of the outside surface, or the entire outside surface, as desired.

The substrate body 26 may be formed from any suitable material. In some aspects, the substrate body 26 may be formed from an elastomeric material comprising polyurethane, nitrile rubber, styrene-butadiene rubber, isobutylene-isoprene rubber, polychloroprene, polyisoprene, natural rubber, or a combination thereof. In some aspects of the disclosure, the substrate body may be formed from an elastomeric material such as natural rubber, which is typically provided as a natural rubber latex. In other embodiments, the elastomeric material may include nitrile butadiene rubber, and in particular, may include carboxylated nitrile butadiene rubber. While articles formed from natural rubber and nitrile rubber are described in detail herein, it should be understood that any other suitable polymer or combination of polymers may be used with the present invention. For instance, the substrate body may be formed from a styrene-ethylene-butylene-styrene (S-EB-S) block copolymer. In some embodiments, the body may be formed from two or more elastomeric materials. For instance, the body may be formed from two or more S-EB-S block copolymers, such as those described in U.S. Pat. Nos. 5,112,900 and 5,407,715 to Buddenhagen et al., both incorporated herein by reference in their entirety. In other embodiments, the elastomeric material may include a styrene-isoprene-styrene block copolymer, styrene-butadiene-styrene block copolymer, styrene-isoprene block copolymer, styrene-butadiene block copolymer, synthetic isoprene, chloroprene rubber, polyvinyl chloride, silicone rubber, or a combination thereof. In yet other embodiments, the substrate body may be formed of a non-elastomeric material such as poly (vinyl-chloride).

A glove is formed on a hand-shaped mold 100, termed a “former”. The former may be made from any suitable material, such as glass, metal, porcelain, ceramic, or the like. The surface of the former defines at least a portion of the surface of the glove to be manufactured. In particular, the surface of the former may define the outside surface of the article 20.

The mold 100 or former may include one or more surface features imprinted thereon. For instance, the mold 100 may have a generally smooth former surface 102 and one or more shapes 104 imprinted in the surface 102. The shapes 104 may be debossed in the surface 102 to form one or more depressed regions relative to the surface 102. As will be described in further detail below, when the mold 100 is used to form the elastomeric article 20, the second surface 24 of the elastomeric article may be considered a “mold side” and may take on the shape of the mold 100 including the surface 102 and the one or more shapes 104, while the first surface 22 opposite the mold 100 may be considered a “free side”. In this manner, the shapes 104 are embossed on the second surface 24 of the elastomeric article 20. In other words, the second surface 24 includes a generally smooth main surface 30 and has one or more embossed shapes 32 protruding from the main surface 30. The embossed shapes 32 may be a mirrored shape of the one or more shapes 104 imprinted on the mold 100. The mold 100 further includes a smooth section 106 free of embossed shapes 106.

In some aspects of the disclosure, the one or more shapes 104 may include one or more geometric shapes, one or more alphabetical letters, one or more Arabic numerals, and/or a combination thereof. In some aspects, the one or more shapes 104 may form a pattern on the surface 102 of the mold 100. For instance, the pattern may be a repeating pattern and/or a non-repeating or an irregular pattern.

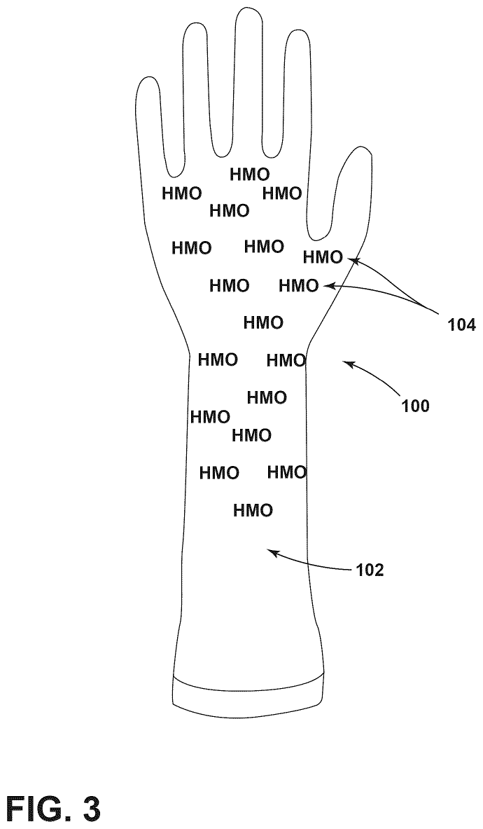

As illustrated in FIG. 1, the glove of the present disclosure may be desired to include a logo, such as an alphabetical logo or an acronym, representing the source of the glove or the intended use of the glove. The embossed shapes 32 may represent the desired logo. On the mold 100, the imprinted shapes 104 are formed as a mirror (mirror image) of the desired logo, as shown in FIG. 3.

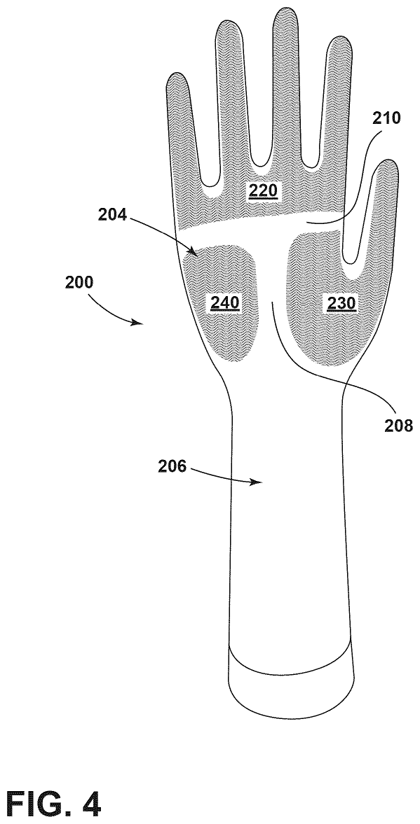

FIG. 4 illustrates a mold 200 having another example design of imprinted shapes 204. In particular, the imprinted shapes 204 include a pattern of wavy or curved lines extending in a generally horizontal direction across the hand portion of a glove. The mold 200 further includes a smooth section 206 free of imprinted shapes 204. In the glove mold 200, imprinted shapes 204 may extend across one or more, e.g., all, of the finger surfaces of the mold 200. In the glove mold 200, imprinted shapes 204 may extend across at least a portion of a palm of the mold 200. In some aspects, the smooth section 206 may include a portion of the palm of the mold 200, e.g. in anatomical locations which may not contribute to a user's grip. For example, as shown in FIG. 4, the mold 200 may include a generally vertical section 208 corresponding with a middle of a user's palm. The mold 200 may additionally or alternatively include a generally horizontal section 210 corresponding with a section of a user's palm below the metacarpophalangeal joints where the fingers connect to the hand.

For example, as shown in FIG. 4, the mold 204 may include a continuous imprinted section 220 extending across a side of one or more of the fingers to the palm side of the glove and over the metacarpophalangeal joints. The mold 204 may further include a continuous imprinted section 230 extending across a side of the thumb and across at least a portion of the palm section. The imprinted sections 220 and 230 may be separated by the horizontal smooth section 210. The mold 204 may further include an outer palm imprinted section 240 away from the thumb and fingers of the mold 200. The outer palm imprinted section 240 may be separated from the imprinted section 220 by the horizontal smooth section 210, and may be separated from the imprinted section 230 by the vertical smooth section 208.

Where the mold 100, 200 may form an ambidextrous glove, i.e., a glove to be worn on either the right hand or the left hand, the mold 100, 200 may have generally identical sides such that a palm side and a dorsal side of a glove may be generally identical. In some aspects, the smooth section 106, 206 may include side surfaces of the mold 100, 200 between the palm side and the dorsal side, and/or along sides of the fingers, such that the side surfaces of a glove formed by the mold 100, 200 are free of the embossed shapes 32.

The present inventors have found that the inclusion of the shapes 32 on the second surface 24 of the elastomeric article, e.g., external surface of a glove, may improve the grip of a user wearing the glove during use such as holding or operating a tool or instrument. Without intending to be bound by theory, the present inventors have found that the shapes 32 protruding from the surface of the glove may augment the frictional force of a user's grip, thereby improving the grip as compared to existing gloves having entirely uniform smooth external surfaces. In particular, forming the shapes 32, e.g., one or more geometric shapes, one or more alphabetical letters, one or more Arabic numerals, and/or a combination thereof, on a palm side of the glove may augment frictional force of the user's grip.

After the elastomeric article layer formulation(s) are compounded, the formulation(s) can be used in a coagulant dip-coating process to form an elastomeric glove. In general, the glove is formed by dipping the former into a series of compositions as needed to attain the desired glove characteristics. The glove may be allowed to solidify between layers. Any combination of layers may be used, and although specific layers are described herein, it should be understood that other layers and combinations of layers may be used as desired.

Where a coagulant based process is used, as in the case of forming a natural rubber glove, the former is first conveyed through a preheated oven to evaporate any water present from cleaning the former. The former is then dipped into a bath (a first solution) typically containing a coagulant, a powder source, a surfactant, and water. The residual heat evaporates the water in the coagulant mixture leaving, for example, calcium nitrate, calcium carbonate powder, and surfactant on the surface of the former. The coagulant may contain calcium ions (e.g., calcium nitrate) that enable a polymer latex, for example, a natural rubber latex or a nitrile rubber latex, to deposit onto the former. The powder may be calcium carbonate powder, which aids release of the completed glove from the former. The surfactant provides enhanced wetting to avoid forming a meniscus and trapping air between the form and deposited latex, particularly in the cuff area. However, any suitable coagulant composition may be used, including those described in U.S. Pat. No. 4,310,928 to Joung, incorporated herein in its entirety by reference.

The coated former is then dipped into a latex containing an elastomeric formulation that forms the substrate body. In some embodiments, the elastomeric material includes natural rubber, which may be supplied as a compounded natural rubber latex. Thus, the bath may contain, for example, compounded natural rubber latex, stabilizers, antioxidants, curing activators, organic accelerators, vulcanizers, and the like. The stabilizers may include phosphate-type surfactants. The antioxidants may be phenolic, for example, 2,2′-methylenebis(4-methyl-6-t-butylphenol). The curing activator may be zinc oxide. The organic accelerator may be dithiocarbamate. The vulcanizer may be sulfur or a sulfur-containing compound. To avoid crumb formation, the stabilizer, antioxidant, activator, accelerator, and vulcanizer may first be dispersed into water by using a ball mill and then combined with the natural rubber latex.

During the dipping process, the coagulant on the former causes some of the elastomeric material to become locally unstable and coagulate onto the surface of the former. The elastomeric material coalesces, capturing the particles present in the coagulant composition at the surface of the coagulating elastomeric material. The former is withdrawn from the bath of elastomeric material and the coagulated layer is permitted to fully coalesce, thereby forming the substrate body. The former is dipped into one or more latex baths a sufficient number of times to attain the desired glove thickness. In some embodiments, the substrate body may have a thickness of from about 0.004 inches (0.1 millimeters) to about 0.012 inches (0.3 millimeters). For instance, when the article 20 is a glove, the thickness of the substrate body 26 may be measured in a palm region of the glove and/or a dorsal side region of the glove.

The former is then dipped into a leaching tank in which hot water is circulated to remove the water-soluble components, such as residual calcium nitrates and proteins contained in the natural rubber latex. The glove is then dried on the former to solidify and stabilize the substrate body. It should be understood that various conditions, process, and materials may be used to form the substrate body.

Other layers may be formed by including additional dipping processes. Such layers may be used to impart additional attributes to the glove. When these processes are complete, the former may then optionally undergo an additional coating process to form an interior or donning layer of the glove. It should be understood that any process may be used to form the donning layer, such as dipping, spraying, immersion, printing, tumbling or any other suitable technique.

When all of the desired polymer layers have been formed and the glove is solidified, the former or mold 100 may be transferred to a stripping station where the article, e.g., glove, is removed from the former. The stripping station may involve automatic or manual removal of the article from the former. For example, in one embodiment, the glove is manually removed and turned inside out (i.e., inverted) as it is stripped from the former. When the article is inverted or turned inside out, the second surface 24 of the article 20 is exposed and forms the outside surface of the article. In this manner, the shapes 32 formed on the second surface 24 are exposed and may become visible to a user.

In some aspects of the present disclosure, a visual marking may be printed on the second surface 24. For instance, a visual marking corresponding to the shapes 32 may be printed on the second surface 24. In this manner, the shapes 32 may be provided with a color distinct from a color of the elastomeric article 20 to enhance the visual distinction between the shapes 32 and the rest of the elastomeric article 20. In some aspects, the visual marking may be printed with a color contrasting the color of the elastomeric article. In some aspects, the visual markings may be provided with different colors on a single elastomeric article 20. Additionally, in some aspects, the visual markings may not correspond to the shapes 32. For instance, the visual markings may take the same or similar form as the shapes 32 but may be printed in different locations on the elastomeric article 20, e.g., separate from the location of each of the shapes 32. In other aspects, the visual markings may include a different design, word, logo, or otherwise visual appearance compared to the shapes 32 formed in the elastomeric article 20.

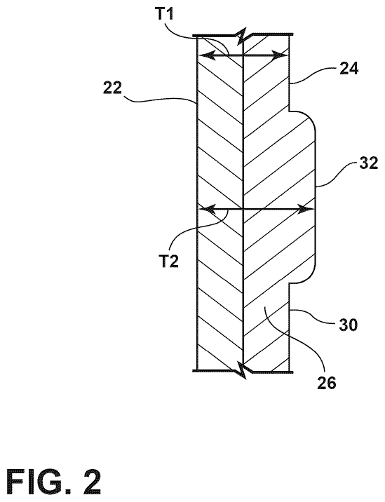

As illustrated in FIG. 2, the elastomeric article 20 may have a thickness extending between the first surface 22 and the second surface 24. The thickness may be greater in a portion of the article 20 including the embossed shape 32, as shown in FIG. 2, where T2 (extending from the first surface 22 to the embossed shape 32) is greater than the thickness T1 (extending from the first surface 22 to the main surface 30 of the second surface).

On the first surface 22 of the article, opposite the second surface 24 including the embossed shape 32, the first surface 22 may form a uniform surface. Stated differently, the first surface 22 may not include any depressions, indentations, or embossed shapes from the one or more shapes 104 of the mold 100. Instead, the first surface 22 may have a smooth and uniform surface free from surface indentations. In this manner, particularly when the article 20 is in the form of a glove as shown in FIG. 1, the inner side or donning side of the glove may provide a smooth and comfortable tactile surface contacting the user's hand.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. An elastomeric article comprising:
a body having a first side and a second side opposite the first side; the body being formed from an elastomeric material, the elastomeric material comprising polyurethane, nitrile rubber, styrene-butadiene rubber, isobutylene-isoprene rubber, polychloroprene, polyisoprene, natural rubber, or a combination thereof;
wherein the first side comprises a uniform surface, and the second side comprises a surface having an embossed shape extending therefrom in a direction opposite the first side of the body.

2. The elastomeric article of claim 1, wherein the surface of the second side comprises a repeating pattern of the embossed shape.

3. The elastomeric article of claim 1, wherein the body forms a glove.

4. The elastomeric article of claim 3, wherein the first side forms an inside surface of the glove and the second side forms an outside surface of the glove.

5. The elastomeric article of claim 4, wherein the surface of the second side comprises a repeating pattern of the embossed shape.

6. The elastomeric article of claim 5, wherein the repeating pattern of the embossed shape extends on both a palm side of the outside surface of the glove and a dorsal side of the outside surface of the glove.

7. The elastomeric article of claim 4, wherein the glove has a palm region thickness ranging from about 0.01 millimeters to about 6 millimeters.

8. The elastomeric article of claim 1, wherein the embossed shape comprises at least one alphabetical letter.

9. The elastomeric article of claim 8, wherein the at least one alphabetical letter comprises a plurality of letters forming a word or an acronym.

10. The elastomeric article of claim 1, wherein the uniform surface of the first side is smooth.

11. The elastomeric article of claim 10, wherein the uniform surface is free from any embossed or debossed shapes.

12. The elastomeric article of claim 1, wherein the body comprises a first layer and a second layer, wherein the first layer forms the first side of the body and the second layer forms the second side of the body.

13. The elastomeric article of claim 1, wherein the body has a first thickness extending from the first side to the second side, wherein the body has a second thickness extending from the first side to the embossed shape of the second side, the second thickness being greater than the first thickness.

14. A process for making an elastomeric article, the process comprising steps of:
providing a mold having a former surface, the mold having a shape debossed in the former surface;
dipping the mold into a first solution comprising a first powder free coagulant;
dipping the mold into a first elastomeric formulation comprising a first elastomeric material to form a first layer; and
curing the first elastomeric formulation to form the elastomeric article, the elastomeric article having a mold side in contact with the mold and a free side opposite the mold side; and
removing the elastomeric article from the mold by inverting the elastomeric article such that the free side of the elastomeric article forms an inside surface of the elastomeric article and the mold side of the elastomeric article forms an outside surface of the elastomeric article, the mold side of the elastomeric article comprising a surface and the shape embossed on the surface.

15. The process of claim 14, wherein after the step of dipping the mold into the first elastomeric formulation, the process includes steps of dipping the mold into a second solution comprising a second powder free coagulant; and
dipping the mold into a second elastomeric formulation comprising a second elastomeric material to form a second layer.

16. The process of claim 14, wherein the shape debossed in the former surface is a mirror image of the shape embossed on the surface of the elastomeric article.

17. The process of claim 14, wherein the inside surface of the elastomeric article comprises a uniform surface.

18. The process of claim 17, wherein the uniform surface is free from any embossed or debossed shapes.

19. The process of claim 14, further comprising a step of printing a visual marking on the shape embossed on the surface.

20. The process of claim 14, further comprising a step of printing a visual marking on the free side of the elastomeric article.
